(19) United States
(12) Patent Application Publication
(10) Pub. No.: US 2013/0018028 A1
(43) Pub. Date: Jan. 17, 2013

(54) Bi-(indole-2-aceto)-iron II (Ferrous Indole Acetate)

(71) Applicant: Christine Maher Farag, Lakeland, FL (US)

(72) Inventor: Christine Maher Farag, Lakeland, FL (US)

(21) Appl. No.: 13/184,502

(22) Filed: Jul. 16, 2011

(51) Int. Cl. A61K 31/555; A61P 27/02; A61P 29/00; A61P 17/06; C07F 15/02; A61P 35/00

(52) U.S. Cl. 514/186; 548/403

(57) ABSTRACT

Bi-(indole-2-aceto)-iron II, also named ferrous bis(indole acetate), or a pharmaceutically acceptable salt thereof, wherein: —Fe (Iron) is substituted by another transition metal capable of bonding to two molecules of indole acetate is synthesized for medicinal purposes including treatment of solid tumor cancers and other vascular proliferative disorders. This invention also extends to the substitution of the ligands by one or more of the structural analogues of indole acetetate disclosed in this application. In certain aspects, the compositions of the invention are capable of generating both a vascular targeting effect and tumor cell cytotoxicity (e.g. by oxidative stress) in order to achieve an enhanced anti-tumor response.

The earliest known cases of cancer hails from the earliest civilizations with documentation going as far as scrolls found in ancient Egypt to a plague of tumors in the old testament. The disease carries with it a myriad of characteristics deriving from any one cell in a mammal's body, but is universally committed to the overproduction of mitochondrion and a lack of telomere shortening after cell division typically found in normal cell division. Because cancer cells resemble the normal cell physiologically, effective selective treatment of cancer has been nonexistent. The only hope traditional medical treatments have to offer is the hope that proliferative cells will pick up the chemotherapueutic agent at large enough doses before normal cells are heavily affected and eventually killed. Treatment with this ideology is found to be more difficult with solid tumors where case layers create an obstacle for the chemotherapeutic agent reuptake. Moreover the instability of tumor cells over time lead to drug resistance to standard therapeutic regimens.

Conventional cancer chemotherapy and radiation are toxic to growing cancer cells but lacks complete specificity. Thus normal tissues are rendered to negative side effects thereby limiting the dose that can be administered. Therefore the exposure of the cancerous tumors to conventional therapies, and in turn the effectiveness of the such therapies, are limited. There is a need for drugs that target the tumor more selectively.

Many solid tumors exhibit oxidative stress due to overproduction of reactive oxygen species. Reactive oxygen species are a group of molecules formed by the incomplete one-electron reduction of oxygen. While these molecules have gained much attention as carcinogens, they also have shown potential as selective cytotoxic agents to cancer. These molecules have been discovered to be responsible for redox signaling which can transduce signals upstream to mitochondria to initiate apoptosis. This reaction cascade reflects a specific aim for drug design in the cancer and other cell proliferative disorders. The hypoxic environment cancer cells provide is selective for activation of ROS and subsequently apoptosis. The concept becomes clear and thus the invention, including structural analogues, is cytotoxic under hypoxic conditions where it is activated by a singlet oxygen moiety and/or ultraviolet light in order to instigate a reaction cascade with the end result being cell apoptosis or inhibition.

The invention pertains to FIG. 2 with chemical name Bi-(indole-2-aceto)-iron II, its structural analogues, a pharmaceutically acceptable salt, or solvate thereof may be used to treat proliferative disorders in which too many of some type of cell are produced. For example, leukemia is a proliferative disorder characterized by an abnormal proliferation i.e., overproduction of white blood cells. Other diseases in which this compound might have medical implications include those involving cellular degradation due to reactive oxygen species productions. Possible structural analogues are examined and illustrated by FIG. 1 which shows iron coordinately bonded to two molecules of indole with inter changeable R groups labeled R₁-R₇ and R′₃ extending forth. In the case of Bi-(indole-2-aceto)-iron II, all R groups are hydrogen as seen in FIG. 2. There are two ligands of indole acetate that coordinately bond to the central metal atom iron represented as Fe. Indole acetate is the conjugate base of indole acetic acid and has no hydrogen on the single bonded oxygen on the corboxylate group. Therefore the negatively charged oxygen atom forms a coordination bond with iron. Examples of the disorders that can be treated with the invention or structural analogues thereof include, prevented, ameliorated or disorders, whose incidence can be reduced, include cancer, rheumatoid arthritis, psoriatic lesions, diabetic retinopathy or wet age-related macular degeneration.

### BRIEF DESCRIPTION OF DRAWINGS

FIG. 1 serves as a skeletal structure from which structural analogues may be constructed through the substitution of functional groups labeled R₁-R₇ and R′₃ outlined in this patent particularly in the Claims and Description portions. Interchangeable functional groups have been assigned R₁-R₇ and R′₃ notation according to prioritization guidelines recognized in the chemistry field. FIG. 2 is the chemical structure of the invention, Bi-(indole-2-aceto)-iron II. In FIG. 2, the functional groups R₁-R₇ and R′₃ are hydrogen. The possible structures delineated in this patent and illustrated in both FIG. 1 and FIG. 2 as well as their pharmaceutical salts and solvents are the invention(s) that this application refers to.

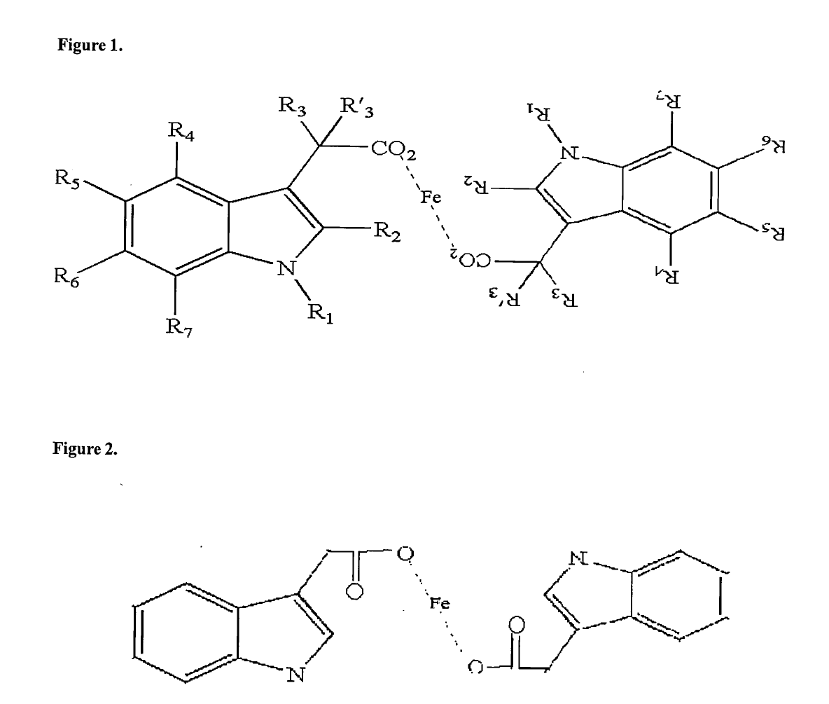

According to a further aspect of the invention known as Bi-(indole-2-aceto)-iron II, and its structural analogues, or a pharmaceutically acceptable salt or solvate thereof, there is an application for treatment of the human or animal body by therapy, particularly for ameliorating or reducing the incidence of a proliferative disorder as defined above in a patient, in which the method comprises of administering to said patient an effective amount of a Bi-(indole-2-aceto)-iron II, structural analogues, or a pharmaceutically acceptable salt thereof. Structural analgoues are achieved through stubstitution of the the functional groups labeled R₁-R₇ and R′3 as seen in FIG. 1. Substitution are explored in the following paragraphs.

A feature of Bi-(indole-2-aceto)-iron II, its structural analogues, pharmaceutically acceptable salts, or solvates thereof, is for use as a medicament, particularly for treatment of the human or animal body.

A further aspect of Bi-(indole-2-aceto)-iron II, its structural analogues, a pharmaceutically acceptable salt, or solvate thereof, involves the manufacture of a medicament for use in the therapy for a warm-blooded animal, like human beings, who suffer from a proliferative disease such as cancer. Bi-(indole-2-aceto)-iron II, its structural analogues, a pharmaceutically acceptable salt, or solvate thereof maybe manufactured as medicament for use in the treatment of the human or animal body, for the prevention or treatment of a said proliferative disorder.

Bi-(indole-2-aceto)-iron II, its structural analogues, a pharmaceutically acceptable salt, or solvate thereof, may be administered as a sole therapy or in combination with other treatments. For the treatment of solid tumors, Bi-(indole-2-aceto)-iron II, its structural analogues, a pharmaceutical acceptable salt or solvate thereof may be administered in combination with radiotherapy or in combination with other anti-tumour substances for example those selected from mitotic inhibitors, for example vinblastine, vincristine, vinorelbine, paclitaxel and docetaxel; alkylating agents, for example cisplatin, carboplatin, oxaliplatin, nitrogen mustard, melphalan, chlorambucil, busulphan and cyclophosphamide; antimetabolites, for example 5-fluorouracil, cytosine arabinoside, gemcitabine, capecitabine, methotrexate and hydroxyurea; intercalating agents for example adriamycin and bleomycin; enzymes, for example aspariginase; topoisomerase inhibitors for example etoposide, teniposide, topotecan and irinotecan; thymidylate synthase inhibitors for example raltitrexed; biological response modifiers for example interferon; antibodies for example edrecolomab, cetuximab, bevacizumab and trastuzumab; receptor tyrosine kinase inhibitors for example gefitinib, imatinib and erlotinib; and anti-hormones for example tamoxifen, anastrazole, exemestane and letrozole. Such combinations of treatment may involve simultaneous or sequential application of the individual components of the treatment.

For prophylaxis and treatment of disease, Bi-(indole-2-aceto)-iron II, its structural analogues, a pharmaceutically acceptable salt, or solvate thereof, may be administered as pharmaceutical compositions selected with regard to the intended route of administration and standard pharmaceutical practice. Such pharmaceutical compositions may take a form suitable for oral, buccal, nasal, topical, rectal or parenteral administration and may be prepared in a conventional manner using conventional excipients. For example for oral administration the pharmaceutical compositions may take the form of tablets or capsules. For nasal administration or administration by inhalation the compounds may be conveniently delivered as a powder or in solution. Topical administration may be as an ointment or cream and rectal administration may be as a suppository. For parenteral injection, including intravenous, subcutaneous, intramuscular, intravascular or infusion, the composition may take various forms such as a sterile solution, suspension or emulsion.

The dose of the Bi-(indole-2-aceto)-iron II, its structural analogues, a pharmaceutically acceptable salt, or solvate thereof, required for the prophylaxis or treatment of a particular condition will vary depending on the compound chosen, the route of administration, the form and severity of the condition and whether the compound is to be administered alone or in combination with another drug. Thus the precise dose will be determined by the administering physician but in general daily dosages may be in the range 0.001 to 100 mg/kg preferably 0.1 to 10 mg/kg. Typically, daily dosage levels are from 0.05 mg to 2 g, for example from 5 mg to 1 g.

Typically, the disorder is cancer. Preferably the cancer is a hypoxic cancer. A hypoxic cancer is a cancer wherein cancerous cells are in a hypoxic environment. Most preferably, the cancer is a solid tumour or leukaemia. Typically the leukaemia is leukaemia involving the spleen or bone marrow or is childhood acute lymphoblastic leukaemia. And typically, the solid tumour is a testicular or a lung tumour.

Bi-(indole-2-aceto)-iron II, its structural analogues, a pharmaceutically acceptable salt, or solvate thereof, is for use in a method of treatment of the human or animal body by therapy, particularly for ameliorating or reducing the incidence of a proliferative disorder as defined above in a patient. The method involves administering to a said patient an effective amount of Bi-(indole-2-aceto)-iron II, its structural analogues, a pharmaceutical acceptable salt or solvate thereof.

Structural analogues of Bi-(indole2-aceto)-iron II may be prepared by a number of processes as generally described below. In the following process description, the symbols Ar, R₁, R₂, R₃ and R₄ when used in the formulae depicted are to be understood to represent those groups described above in relation to FIG. 1 unless otherwise indicated. In the schemes described below it may be necessary to employ protecting groups that are then removed during the final stages of the synthesis. The appropriate use of such protecting groups and processes for their removal will be readily apparent to those skilled in the art.

Bi-(indole-2-aceto)-iron II with the chemical structure depicted in FIG. 2 can be prepared by refluxing ferrous sulfate with two equivalents of indole acetic acid sodium salt in a polar solvent such as water. It is easier for solutions of the reactants to be prepared separately in a polar solvent like nanopure water and then combined. Small amounts of an acid may be added to the indole acetic acid sodium salt, or its structural analogues, to decrease the pH in order to increase solubility. Too much acid might precipitate iron forming ferric hydroxide. Caution must be taken to avoid this. Alcohol may also be added to a solution of ferrous sulfate, or its structural analogue to increase solubility. The two solutions may be combined and mixed until the desired product forms. The next step would be to remove the solvent. There are many ways to do this including, but not limited to evaporation, distillation, and reduced pressure evaporation just to name a few. The solvent was removed in this case via rotary evaporation. The crude product is further purified using acetone to remove undesired salts such as sodium sulfate or sodium chloride. The final product dissolved in acetone was then placed in a rotary evaporator to remove the acetone. The final product is ferrous bis(indole acetate) or Bi-(indole-2-aceto)-iron II.

Structural analogues of indole acetic acid sodium salt may be made yielding the product mentioned in claims 1-13. Also a different salt combination or no salt at all may be used such as indole acetic acid potassium salt or indole acetic acid. Different combinations are rendered through the scope of the chemical structure presented in FIG. 1.

Substitutions for ferrous sulfate is also implied in this patent keeping in line with the fact that they yield the products mentioned in claims 1-15. Examples include Cuprous sulfate, ferrous chloride, etc. The metal salt should be a transition metal that when bonded to two molecules of indole acetate, or its structural analogues mentioned in claims 1-15, provide a cooxidizing mechanism suitable for the fenton reaction under hypoxic conditions.

As previously stated FIG. 1 provides a skeletal structure with functional groups labeled R₁-R₇ and R′₃. R₁, R₂, R₃ and R′₃ are independently selected from H or lower alkyl; and R₄, R₅, R₆ and R₇ are independently selected from H, electron withdrawing groups (such as F, Cl, Br, I, OCF₃), carboxyl groups, acetal groups, electron deficient aryl groups), lower alkyl groups, lower alkoxy groups, aryl groups or aryloxy groups, wherein at least one of R₄, R₅, R₆ and R₇ is selected from an electron withdrawing group. See FIG. 1.

Electron-withdrawing' groups are those groups which reduce the electron density in other parts of the molecule. Those groups suitable for of functional groups R₁-R₇ and R′₃ include F, Cl, Br, I, OCF₃, carboxyl groups, acetal groups and electron deficient aryl groups. The preferred electron withdrawing groups are F, Cl, Br and I, of which F, Cl and Br are most preferred.

Lower alkyl' in this application means a group having from 1 to 7 carbon atoms, which may be aliphatic or alicyclic, or a combination thereof and which may be saturated, partially unsaturated, or fully unsaturated. This group may bear one or more substituents, selected from halo e.g., F, Cl, Br, I, preferably F, Cl, Br, carboxyl, acetal, aryl, aryloxy and alkoxy.

Examples of saturated linear lower alkyl groups include, but are not limited to, methyl, ethyl, n-propyl, n-butyl, and n-pentyl(amyl).

Examples of saturated branched lower alkyl groups include, but are not limited to, iso-propyl, iso-butyl, sec-butyl, tert-butyl, and neo-pentyl.

Examples of saturated alicyclic(carbocyclic) lower alkyl groups (also referred to as —C₃₋₇ cycloalkyl ∥ groups) include, but are not limited to, unsubstituted groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methycyclopentyl, dimethycyclopentyl, methylcyclohexyl, dimethylcyclohexyl, cyclopropylmethyl and cyclohexylmethyl. See FIG. 1.

Examples of unsaturated lower alkyl groups which have one or more carbon-carbon double bonds (also referred to as —C₂₋₇ alkenyl ∥ groups) include, but are not limited to, ethenyl (vinyl, —CH═CH₂), 2-propenyl (allyl, —CH—CH═CH₂), isopropenyl (—C(CH₂)═CH₂), butenyl, pentenyl, and hexenyl. See FIG. 1.

Examples of unsaturated lower alkyl groups which have one or more carbon-carbon triple bonds (also referred to as —C₂₋₇ alkynyl ∥ groups) include, but are not limited to, ethynyl(ethinyl) and 2-propynyl(propargyl). See FIG. 1.

Examples of unsaturated alicyclic(carbocyclic) lower alkyl groups which have one or more carbon-carbon double bonds (also referred to as —C3-cycloalkenyl ∥ groups) include, but are not limited to, unsubstituted groups such as cyclopropenyl, cyclobutenyl, cyclopentenyl, and cyclohexenyl, as well as substituted groups (e.g. groups which comprise such groups) such as cyclopropenylmethyl and cyclohexenylmethyl. See FIG. 1.

Carboxyl' in this application means a group of structure —C(═O)—, and includes carboxylates, acyl groups, amides and acyl halides.

Carboxylates ‘means groups of structure —C(═O)OR, where R is H or a carboxyl substituent, for example, a lower alkyl group or a C₅₋₂₀ aryl group, preferably a lower alkyl group. Examples of carboxyl groups include, but are not limited to —C(═O)OH, —C(═O)OCH₃, —C(═O)OCH2CH₃, —C(═O)OC(CH₃)₃, and —C(═O)OPh. See FIG. 1.

Acyl' in this application means a group of structure —C(═O)R, where R is H or an acyl substituent, for example, a lower alkyl group or a C₅₋₂₀ aryl group, preferably a lower alkyl group. Examples of acyl groups include, but are not limited to, —C(═O)H (formyl), —C(═O)CH₃ (acetyl), —C(═O)CH₂CH₃ (propionyl), —C(═O)C(CH₃)₃ (butyryl), and —C(═O)Ph (benzoyl, phenone). See FIG. 1.

Amides' in this application means groups of structure —C(═O)NR₁R₂, wherein R₁ and R₂ are independently amino substituents, for example, hydrogen, a lower alkyl group (also referred to as C₁₋₇ alkylamino or di-C₁₋₇ alkylamino), or a C₅₋₂₀ aryl group, preferably H or a C₁₋₇ alkyl group. Examples of amido groups include, but are not limited to, —C(═O)NH₂, —C(═O)NHCH₃, —C(═O)NH(CH₃)₂₁—C(═O)NHCH₂CH₃, and —C(═O)N(CH₂CH₃)₂, as well as amido groups in which R₁ and R₂, together with the nitrogen atom to which they are attached, form a heterocyclic structure as in, for example, piperidinocarbonyl, morpholinocarbonyl, thiomorpholinocarbonyl, and piperazinocarbonyl. See FIG. 1.

Acyilhalide ‘means groups of structure —C(═O)X, wherein X is —F, —Cl, —Br, or —I, preferably —Cl, —Br, or —I.

Acetal' in this application means a structure —C(OR₃)(OR₄)—, where the third substituent is as for a carbonyl group (defined above). R₃ and R₄ are selected from lower alkyl groups, or may together form a divalent alkyl group. See FIG. 1.

Alkoxy' in this application means a group of structure —OR, wherein R is an optionally substituted lower alkyl group, wherein the substituent may include halo (F, Cl, Br, I) and aryl. Examples of alkoxy groups include, but are not limited to, —OCH₃(methoxy), —OCH₂CH₃ (ethoxy), —OC(CH₃)₃(tert-butoxy), —OBn(benzyloxy), and —OCH₂F(fluoromethoxy). See FIG. 1.

Aryl' in this application means a monovalent moiety obtained by removing a hydrogen atom from a ring atom of a C₅₋₂₀ aromatic compound (also known as a C₅₋₂₀ aryl group), said compound having one ring, or two or more rings (e.g. fused), and having from 5 to 20 ring atoms. Preferably, each ring has from 5 to 7 ring atoms. See FIG. 1.

The ring atoms may be all carbon atoms, as in -carboaryl groups ∥, in which case the group may conveniently be referred to as a —C₅₋₂₀ carboaryl ∥ group. See FIG. 1.

Examples of C₅₋₂₀ heterocyclic groups (including C₅₋₂₀ heteroaryl groups) which comprise fused rings include, but are not limited to, those derived from quinoline, isoquinoline, purine (e.g., adenine, guanine), benzimidazole, carbazole, fluorene, phenoxathiin, benzofuran, indole, isoindole, quinoxaline, phenazine, phenoxazine, xanthene, acridine, and phenothiazine. See FIG. 1.

Examples of aryl groups which do not have ring heteroatoms i.e., C₅₋₂₀ carboaryl groups include, but are not limited to, those derived from benzene e.g., phenyl, naphthalene, anthracene, phenanthrene, and pyrene. See FIG. 1.

Alternatively, the ring atoms may include one or more heteroatoms, including but not limited to oxygen, nitrogen, and sulfur, as in -heteroaryl groups. ∥ In this case, the group may conveniently be referred to as a —C₅₋₂₀ heteroaryl ∥ group, wherein —C₅₋₂₀ ∥ denotes ring atoms, whether carbon atoms or heteroatoms. Preferably, each ring has from 5 to 7 ring atoms, of which from 0 to 4 are ring heteroatoms. See FIG. 1.

Examples of C₅₋₂₀ heteroaryl groups include, but are not limited to, C₅ heteroaryl groups derived from furan(oxole), thiophene(thiole), pyrrole(azole), imidazole(1,3-diazole), pyrazole(1,2-diazole), triazole, oxazole, isoxazole, thiazole, isothiazole, oxadiazole, and oxatriazole; and C₆ heteroaryl groups derived from isoxazine, pyridine(azine), pyridazine(1,2-diazine), pyrimidine(1,3-diazine; e.g. cytosine, thymine, uracil), pyrazine(1,4-diazine), triazine, tetrazole, and oxadiazole(furazan). See FIG. 1.

Examples of C₅₋₂₀ heterocyclic groups (including C₅₋₂₀ heteroaryl groups) which comprise fused rings, include, but are not limited to, those derived from quinoline, isoquinoline, purine (e.g., adenine, guanine), benzimidazole, carbazole, fluorene, phenoxathiin, benzofuran, indole, isoindole, quinoxaline, phenazine, phenoxazine, xanthene, acridine, and phenothiazine. See FIG. 1.

Electron deficient aryl groups' in this application means an aryl group which is electron withdrawing, and includes heteroaryl groups. An example of an electron deficient aryl group is para-chlorophenyl.

Aryloxy' in this application means a group of structure —OR, wherein R is an aryl group. An example of an aryloxy group is —OPh(phenoxy). See FIG. 1.

Physiologically functional derivatives of prodrugs include salts, amides and esters. Esters include carboxylic acid esters in which the non-carbonyl moiety of the ester grouping is selected from straight or branched chain C₁₋₆ alkyl (e.g. methyl, n-propyl, n-butyl or t-butyl); or C₃₋₆ cyclic alkyl (e.g. cyclohexyl). Salts include physiologically acceptable base salt, e.g. derived from an appropriate base, such as alkali metal (e.g. sodium, alkaline earth metal (e.g. magnesium) salts, ammonium and NR 14 (where R ∥ is C₁₋₄ alkyl) salts. Other salts include acid addition salts, including the hydrochloride and acetate salts. Amides include non-substituted and mono and di substituted derivatives. Such derivatives may be prepared by techniques known per se in the art of pharmacy.

It is preferred that the balance, or overall effect, of the substituents R₄, R₅, R₆ and R₇ is electron withdrawing. See FIG. 1.

The following preferences for the groups R₁, R₂, R₃, R′₃, R₄, R₅, R₆ and R₇ may be independent of each other or may be in any combination with each other. See FIG. 1.

R₁ and R₂ are preferably selected from H or optionally substituted saturated lower alkyl groups, more preferably optionally substituted saturated linear lower alkyl groups, more particularly methyl or ethyl. The most preferred group for R₁ and R₂ is H. See FIG. 1.

If the lower alkyl group of R₁ is substituted, the substituent is preferably one which aids solubility of the whole compound, such as morpholino or piperazinyl. See FIG. 1.

R′₃ is preferably H. R₃ is preferably selected from H or optionally substituted saturated lower alkyl groups, more preferably optionally substituted saturated linear lower alkyl groups, more particularly methyl or ethyl. The most preferred group for R₃ is H, so that in combination R₃ and R′₃ are both H. See FIG. 1.

It is preferred that one or two, more preferably one, of R₄, R₅, R₆ and R₇ are independently selected from electron withdrawing groups. See FIG. 1.

If one or more of R₄, R₅, R₆ and R₇ are not H or an electron withdrawing groups, they are preferably selected from optionally substituted saturated lower alkyl groups, more preferably optionally substituted saturated linear lower alkyl group and most preferably un-substituted linear lower alkyl groups, more particularly methyl or ethyl. See FIG. 1.

It is most preferred that one of R₄, R₅, R₆ and R₇ is an electron withdrawing group and the rest are H, and in particular that R₅, is the electron withdrawing group. See FIG. 1.

Racemic mixtures of Bi-(indole-2-aceto)-iron II, in and/or its structural analogues, a pharmaceutically acceptable salt, or solvate thereof, in any one combination may also be obtained.

Bi-(indole-2-aceto)-iron II, along with its structural analogues, including pharmaceutically acceptable salts and solvates there of, is activated by a peroxide that induces a fenton reaction yielding the desired reactive oxygen species that have been shown to be cytotoxic to proliferative diseased cells and not normal cells. Hydrogen peroxide is the peroxide of choice for the activation of of Bi-(indole-2-aceto)-iron II or structural analogues mentioned in this patent. Within the scope of chemistry, a peroxide is understood to be a compound containing an oxygen-oxygen single bonded or the peroxide anion ([O—O]₂—). Photodynamic therapy (PDT) involves the use of light to activate molecules in order to produce toxic species. The majority of current and proposed technique's use singlet oxygen as the toxic species derived from the reaction of a photo sensitizer with cellular oxygen. The technique's main drawback is that it does not work in anoxic tumours and that the photosensitizers are not readily excreted by the body, and therefore patients treated with PDT remain sensitive to light for a considerable period after treatment.

As stated earlier, Photodynamic therapy (PDT) involves the use of light to activate molecules in order to produce toxic species. The majority of current and proposed technique's use singlet oxygen as the toxic species derived from the reaction of a photo sensitizer with cellular oxygen. The technique's main drawback is that it does not work in anoxic tumours and that the photosensitizers are not readily excreted by the body, and therefore patients treated with PDT remain sensitive to light for a considerable period after treatment.

In photodynamic therapy, PDT, the direction and width of a laser beam can be controlled with great precision. Therefore, it can act upon a very limited area, minimizing damage to neighboring tissue.

The activation process in PDT can be highly site specific. The direction and width of a laser beam can be controlled with great precision. Therefore, it can act upon a very limited area, minimizing damage to neighboring tissue.

Apoptosis induction in human tumour cells by photoproducts of indole-3-acetic acid sensitized by riboflavin has recently been described (Edwards, A. M., et al. Apoptosis induction in non-irradiated human HL-60 and murine NSO/2 tumor cells by photoproducts of indole-3-acetic acid and riboflavin.

The use of compund Bi-(indole-2-aceto)-iron II, its structural analogues including pharmaceutical acceptable salts and solvate thereof enhances PDT. In PDT, photolysis of photosensitizers P, equation (1), results in the reaction of the generated triplet excited state 3 P* with ground state oxygen, producing toxic singlet oxygen, equation (2). One drawback with conventional PDT is that no toxicity occurs in anoxia. Without wishing to be bound by theory, Bi-(indole-2-aceto)-iron II or its structural analogues illustrated as IAA bellow, are oxidized by 3 P* to generate an indole radical cation, equation (3) which leads to toxic products, with or without the involvement of oxygen. P+hv→3 P* (equation 1) 3 P*+O 2→P+1 O 2 (equation 2) 3 P*+IAA→P+IAA.+ (equation 3) The combination, either in aerobic or anoxic conditions, Bi-(indole-2-aceto)-iron II, or its derivative disclosed in this patent, with a photosensitizer should result in a lower concentration of photosensitizer being needed to achieve the same toxicity. This would reduce the normal tissue damage from light exposure after treatment, which can occur up to several weeks after treatment. The sensitizing effects of IAA derivatives should last only for a few hours, until the IAA is excreted.

The technique of PDT as discussed above can be used by employing a combination of appropriate compounds Bi-(indole-2-aceto)-iron II and those represented in this patent in FIG. 1 and photosensitizers. The preferred wavelength of light used is 500 to 800 nm.

Compounds of FIG. 1 and FIG. 2 in conjunction with photosensitizers activated by light having a wavelength between 500 and 800 nm may be tested in vitro with other suitable forms of activation against panels of different tumour cell types to determine efficacy against such tumour cells. The efficacy of Bi-(indole-2-aceto)-iron II, its structural analogues, a pharmaceutical acceptable salt or solvate thereof against a range of tumour cell types may be used as points of reference for the development of further anti-tumour compounds.

Formulations suitable for parenteral or intramuscular administration include aqueous and non-aqueous sterile injection solutions which may contain anti-oxidants, buffers, bacteriostatis, bacteriocidal antibiotics and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose containers, for example sealed ampoules and vials, and may be stored in a freeze-dried (lyophilized) condition requiring only the addition of a sterile liquid carrier, for example Water for Injection, immediately prior to use. Injection solutions and suspensions may be prepared extemporaneously from sterile powders, granules and tablets of the kind previously described.

It should be understood that in addition to the ingredients particularly mentioned above, the formulations may include other agents conventional in the art having regard to the type of formulation in question. Of the possible formulations, sterile pyrogen-free aqueous and non-aqueous solutions are preferred.

The doses may be administered sequentially, e.g. at hourly, daily, weekly or monthly intervals, or in response to a specific need of a patient. Preferred routes of administration are oral delivery and injection, typically parenteral or intramuscular injection or intratumoural injection. For methods of PDT dermal or topical administration may be preferred, e.g. subcutaneous injection or creams and ointments, and such methods of administration are well known.

The exact dosage regimen will need to be determined by individual clinicians for individual patients and this, in turn, will be controlled by the exact nature of the compound Bi-(indole-2-aceto)-iron II, or its derivative as disclosed in this patent, but some general guidance can be given. Typical dosage ranges generally will be those described above which may be administered in single or multiple doses. Other doses may be used according to the condition of the patient and other factors at the discretion of the physician.

1. A process for treating cancer or a proliferative disorder in a mammals which comprises of administrating an effective amount of Bi-(indole-2-aceto)-iron II represented by the formula Fe(C₁₀H₈NO₂)₂, or its structural analogues. The compound generates reactive oxygen species when combined with a peroxide.

2. The synthesis of M(C₁₀H₈NO₂)₂, where M represents a transition metal, via a salt exchange mechanism. Protocol for synthesizing Bi-(indole-2-aceto)-iron II, also known as Ferrous his (Indole Acetate), comprises of Ferrous Sulfate refluxed with Indole Acetic Acid Sodium Salt in a polar solvent. The desired product is further isolated using acetone in which the crude product is dissolved and thus further purified.

3. Compounds of claims 1-2, or physiologically functional structural analogues thereof, wherein as depicted in R₁, R₂, R₃ and R′₃ are independently selected from lower alkyl; and R₄, R₅, R₆ and R₇ are independently selected from H, electron withdrawing groups (such as F, Cl, Br, I, OCF₃, carboxyl groups, acetal groups, electron deficient aryl groups), lower alkyl groups lower alkoxy groups, aryl groups or aryloxy groups, wherein it least one of R₄, R₅, R₆ and R₇ is selected from an electron withdrawing group, may be used in methods of therapy, particular in treating proliferative disorders or neoplastic diseases.

4. The pharmaceutical composition according to claims 1-2, wherein the electron withdrawing group is selected from the group consisting of F, Cl, Br, I, OCF₃, carboxyl, acetal and electron deficient aryl.

5. The pharmaceutical composition according to claims 1-2, wherein the electron withdrawing group is selected from the group consisting of F, Cl, Br and I.

6. The pharmaceutical composition according to claims 1-2, wherein the balance of the substituents R₄, R₅, R₆ and R₇ is electron withdrawing.

7. The pharmaceutical composition according to claims 1-2, wherein R₁ is independently selected from H or optionally substituted saturated lower alkyl groups.

8. The Pharmaceutical composition according to claims 1-2, wherein R′₃ is H.

9. The pharmaceutical composition according to claims 1-2, wherein R₃ is selected from H or optionally substituted saturated lower alkyl groups.

10. The pharmaceutical composition according to claim 10, wherein R₃, is selected from H, methyl or ethyl.

11. The pharmaceutical composition according to claims 1-2, wherein one or two of R₄, R₅, R₆ and R₇, are independently selected from electron withdrawing groups.

12. The pharmaceutical composition according to claims 1-2, wherein if one or more of R₄, R₅, R₆ and R₇, are not H or an electron withdrawing groups, they are selected from optionally substituted saturated lower alkyl groups such as H, ethyl, or methyl group.

13. The pharmaceutical composition according to claim 12, wherein those of R₄, R₅, R₆ and R₇, which are not H or an electron withdrawing group are selected from H, methyl or ethyl group.

14. The pharmaceutical composition according to claims 1-2, wherein one of R₄, R₅, R₆ and R₇, is an electron withdrawing group and the rest are H.

15. A method for selectively reducing blood flow to a tumor region by forming a ROS species in a patient suffering from cancer through administration of a compound stated in claims 1-15 to a said patient.

16. A method of inhibiting the proliferation of tumor cells in a patient suffering from cancer, involving the administration of a compound mentioned in claims 1-15 to the patient of an effective amount.

17. A method of reducing blood flow in a patient suffering from a vascular proliferative disorder, involving administering to the patient an effective amount of a compound mentioned in claims 1-15.

18. A kit comprising of; (a) a pharmaceutical composition comprising tablets, each comprising a compound of any one of claims 1-15 and a pharmaceutically acceptable carrier, (b) a packaging material enclosing said pharmaceutical composition, (c) pharmaceutical grade peroxide (optional) and (d) instructions for use of said pharmaceutical composition in the treatment of a subject in need thereof.

19. For the avoidance of doubt, the invention extends to the compounds mentioned in claim 1-15 in pharmaceutically acceptable solvate form.

20. A pharmaceutical composition comprising a compound in claims 1-15 in combination with a pharmaceutically acceptable carrier.

21. Method according to claim 1, wherein the proliferative disorder is cancer, rheumatoid arthritis, psoriatic lesions, neoplastic disorder diabetic retinopathy or wet age-related macular degeneration. The proliferative disorder also encompasses hypoxic disorders.

22. The combination of a compound from claims 1-15 with a peroxide to generate reactive oxygen species that may be used in proliferative and neoplastic disorders and by the delivery mechanisms ascribed in claims 16-26.

23. The compound in claims in 1-15 may also be activated by UVB to be used.
